(19) United States
(12) Patent Application Publication
(10) Pub. No.: US 2023/0200900 A1
(43) Pub. Date: Jun. 29, 2023

(54) MEDICAL DEVICE AND METHOD FOR ACCESSING THE PERICARDIAL SPACE

(71) Applicant: Boston Scientific Medical Device Limited, Galway (IE)

(72) Inventor: Brock Miller, Mississauga (CA)

(21) Appl. No.: 18/170,791

(22) Filed: Feb. 17, 2023

Related U.S. Application Data
(63) Continuation of application No. PCT/IB2021/057448, filed on Aug. 12, 2021.
(60) Provisional application No. 63/067,366, filed on Aug. 19, 2020.

(51) Int. Cl. A61B 18/14

(52) U.S. Cl. CPC A61B 18/1492; A61B 2018/00077

(57) ABSTRACT

A medical device includes a guidewire extending between a proximal portion defining a proximal end and a distal portion defining a distal end. The distal portion includes a radially expandable part, and the guidewire is movable between a retracted configuration in which the part is radially retracted and an expanded configuration in which the part is radially expanded. The part is radiopaque.

### CROSS REFERENCE TO RELATED APPLICATIONS

This application is a continuation of PCT/IB2021/057448, filed Aug. 12, 2021, which claims priority to U.S. Provisional Patent Application No. 63/067,366, filed Aug. 19, 2020, both of which are incorporated herein in their entireties.

### FIELD

This document relates to medical devices. More specifically, this document relates to medical devices such as guidewires, and methods for accessing the pericardial space using such medical devices.

### SUMMARY

The following summary is intended to introduce the reader to various aspects of the detailed description, but not to define or delimit any invention.

Medical devices are disclosed. According to some aspects, a medical device includes a guidewire extending between a proximal portion defining a proximal end and a distal portion defining a distal end. The distal portion includes at least one part that is radially expandable. The guidewire is movable between a retracted configuration in which the part is radially retracted and an expanded configuration in which the part is radially expanded. The part is radiopaque.

In some examples, the part includes a plurality of tines. When the guidewire is in the retracted configuration, the tines lie generally flat, and when the guidewire is in the expanded configuration, the tines flex radially outwardly. The tines can be circumferentially spaced apart.

In some examples, the guidewire includes a main wire extending between the proximal portion and the distal portion, and a sleeve on the main wire in the distal portion. The sleeve can include a body and the plurality of tines. The guidewire can further include a retainer on the sleeve, and the retainer can include a plurality of apertures. When the guidewire is in the retracted configuration, the tines can be held within the retainer, and when the guidewire is in the expanded configuration, the tines can extend through the apertures and away from the sleeve.

In some examples, the main wire includes an electrical conductor and an electrically insulative material on the electrical conductor. The guidewire can further include an electrode at the distal end.

In some examples, the guidewire includes a main wire extending between the proximal portion and the distal portion, and the main wire is the radially expandable part. When the guidewire is in the retracted configuration, a section of the main wire can be generally straight, and when the guidewire is in the expanded configuration, the section can be serpentine. For example, when the guidewire is in the expanded configuration, the section can be coiled or waved. The section can be waved in one plane or more than one plane.

In some examples, in the distal portion, the main wire is split into a first section and a second section. When the guidewire is in the retracted configuration, the first section and the second section can lie flat against each other, and when the guidewire is in the expanded configuration, the first section and the second section can be flexed away from each other.

In some examples, the guidewire includes a main wire extending between the proximal portion and the distal portion, and the part includes an inflatable balloon mounted to the main wire in the distal portion. When the guidewire is in the retracted configuration, the balloon can be deflated, and when the guidewire is in the expanded configuration, the balloon can be inflated.

In some examples, the part is biased towards the expanded configuration.

Methods for accessing the pericardial space are also disclosed. According to some aspects, a method for accessing the pericardial space includes: a. creating a puncture in a pericardium; b. with a guidewire in a retracted configuration, advancing a distal portion of the guidewire through the puncture into the pericardial space; c. moving the guidewire to an expanded configuration; and d. with the guidewire in the expanded configuration and in the pericardial space, viewing the distal portion under fluoroscopy.

In some examples, step c. includes flexing tines of the guidewire radially outwardly.

In some examples, step c. includes reshaping a section of the guidewire. Step c. can include moving the section of the guidewire from a straight shape to a serpentine shape. Step c. can include moving the section of the guidewire from the straight shape to a coiled shape or a waved shape.

In some examples, step c. includes flexing a first section of the guidewire away from a second section of the guidewire.

In some examples, step c. includes inflating a balloon of the guidewire.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a side view of an example medical device in the form of a guidewire, showing the guidewire in a retracted configuration;

FIG. 2 is an enlarged side view of the distal portion of the guidewire of FIG. 1, showing the guidewire in the retracted configuration;

FIG. 3 is a cross section taken along line 3-3 in FIG. 2;

FIG. 4 is an enlarged side view of the distal portion of the guidewire of FIG. 1, showing the guidewire in an expanded configuration;

FIG. 5 is an end view of the guidewire of FIG. 1, showing the guidewire in the expanded configuration;

FIG. 6 is a schematic view showing the guidewire of FIG. 1 in use, positioned in the pericardial space, and in the expanded configuration;

FIG. 7 is a side view of the distal portion of another example guidewire, showing the guidewire in an expanded configuration;

FIG. 8 is a side view of the distal portion of another example guidewire, showing the guidewire in an expanded configuration;

FIG. 9 is a side view of the distal portion of another example guidewire, showing the guidewire in a retracted configuration;

FIG. 10 is a side view of the distal portion of the guidewire of FIG. 9, showing the guidewire in an expanded configuration;

FIG. 11 is a side view of the distal portion of another example guidewire, showing the guidewire in an expanded configuration;

FIG. 12 is an end view of the guidewire of FIG. 11, showing the guidewire in the expanded configuration;

FIG. 13 is an end view of another example guidewire, showing the guidewire in an expanded configuration;

FIG. 14 is a side view of the distal portion of another example guidewire, showing the guidewire in an expanded configuration;

FIG. 15 is an end view of the guidewire of FIG. 14, showing the guidewire in the expanded configuration;

FIG. 16 is an end view of another example guidewire, showing the guidewire in an expanded configuration;

FIG. 17 is a side view of the distal portion of another example guidewire, showing the guidewire in a retracted configuration;

FIG. 18 is a side view of the distal portion of the guidewire of FIG. 17, showing the guidewire in an expanded configuration;

FIG. 19 is a side view of the distal portion of another example guidewire, showing the guidewire in a retracted configuration;

FIG. 20 is a side view of the distal portion of the guidewire of FIG. 19, showing the guidewire in an expanded configuration; and

FIG. 21 is an end view of the guidewire of FIGS. 19 and 20, showing the guidewire in the expanded configuration.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are medical devices in the form of guidewires. The guidewires can be used in medical procedures in which the pericardial space is accessed via a puncture in the pericardium. The guidewires can optionally be advanced into the pericardial space after a separate medical device (e.g. a needle) has been used to puncture the pericardium, or the guidewires can create the puncture (e.g. the guidewires can be configured to deliver radiofrequency energy to puncture the pericardium) and then be advanced into the pericardial space. The guidewires are generally configured so that the distal portion thereof includes one or more parts that is both radially expandable and visible under fluoroscopy (i.e. is radiopaque). When the distal portion is advanced into the pericardial space, the part(s) can be expanded, and can generally fill and conform to the contours of the pericardial space. The distal portion can then be viewed under fluoroscopy, to confirm that access to the pericardial spaces has been gained and to gain information about the shape of the pericardial space.

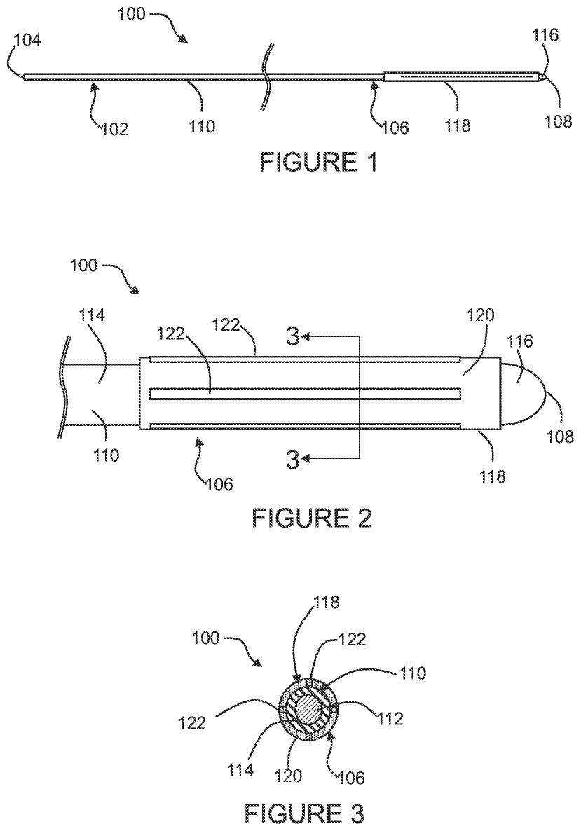

A first example of a guidewire 100 is shown is shown in FIGS. 1 to 6. Referring first to FIG. 1, the guidewire 100 extends longitudinally between a proximal portion 102 defining a proximal end 104 and a distal portion 106 defining a distal end 108. In the example shown, the guidewire 100 is a radiofrequency (RF) puncture guidewire, and includes a main wire 110 extending between the proximal portion 102 and the distal portion 106. Referring to FIGS. 2 and 3, the main wire 110 includes an electrical conductor 112 (visible in FIG. 3) and an electrically insulative material 114 (such as high density polyethylene) on the electrical conductor 112. The electrically insulative material 114 is preferably also lubricious. An electrode 116 (visible in FIG. 2) is at the distal end 108 of the guidewire 100, and is in electrical contact with the electrical conductor 112. The main wire 110 can be connected to an RF generator (not shown) at the proximal end 104, and RF energy can be delivered from the RF generator along the main wire 110 to the electrode 116. The RF energy can then be delivered from the electrode 116 to a tissue with which the electrode 116 is in contact, to puncture the tissue.

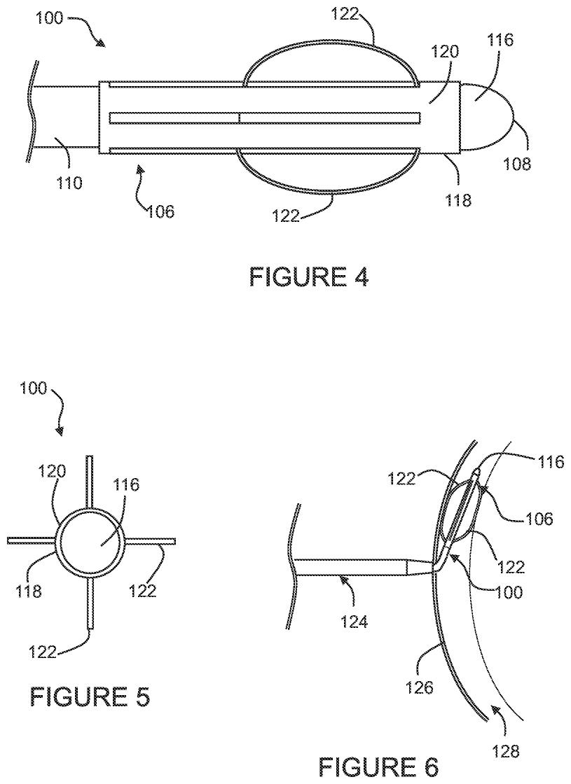

As mentioned above, the distal portion 106 of the guidewire 100 includes one or more parts that are both radially expandable and radiopaque, and the guidewire 100 is movable between a retracted configuration in which the parts are radially retracted and an expanded configuration in which the parts are radially expanded. Referring to FIGS. 2 to 5, in the example shown, the guidewire 100 includes a sleeve 118 that is received on the main wire 110 in the distal portion 106. The sleeve 118 includes a body 120 and a plurality of tines 122 (only two of which are labelled). Each tine 122 serves as a radially expandable part. When the guidewire 100 is in the retracted configuration, as shown in FIGS. 2 and 3, the tines 122 lie generally flat. When the guidewire 100 is in the expanded configuration, as shown in FIGS. 4 and 5, the tines 122 flex radially outwardly.

The tines 122 can in some examples be biased towards the expanded configuration. For example, the sleeve 118 can be fabricated from a shape memory material such as nitinol, and the tines 122 can be laser cut from the sleeve 118 (leaving behind grooves, as shown) and heat treated so that they are biased towards the expanded configuration. Alternatively, the tines 122 can be made from stainless steel. The tines 122 can include a coating of a radiopaque material such as platinum or gold.

Referring to FIG. 6, in use, the guidewire 100 can be advanced towards the heart via an introducer 124. The guidewire 100 can initially be retained in the retracted configuration by the introducer 124, which will press the tines 122 flat. With the tip of the introducer 124 in contact with the heart, and the electrode 116 of the guidewire 100 protruding from with the tip of the introducer 124, RF energy can be delivered from the electrode 116, to create a puncture in the pericardium 126. The guidewire 100 can then be advanced through the puncture and into the pericardial space 128. As the distal portion 106 of the guidewire passes through the puncture and into the pericardial space 128, the guidewire 100 will initially remain in the retracted configuration, due to contact with the introducer 124. As the guidewire 100 continues to be advanced out of the introducer 124 and the tines 122 clear the introducer, the guidewire 100 will automatically move to the expanded configuration, as shown in FIG. 6, with the tines 122 flexed radially outwardly, generally filling the pericardial space 128, and conforming to the shape of the heart. With the guidewire 100 in the expanded configuration, the distal portion 106 of the guidewire 100 can then be viewed under fluoroscopy, both to confirm that access to the pericardial space 128 has been gained, and to gain information about the shape of the pericardial space 128.

In the example shown, the guidewire 100 is biased towards the expanded configuration and automatically moves from the retracted configuration to the expanded configuration upon sufficiently clearing the introducer 124. In alternative examples, the guidewire 100 can be manually moved towards the expanded configuration. For example, the guidewire can include one or more pull-wires (not shown) that can be actuated to cause the tines to flex radially outwardly or to retract.

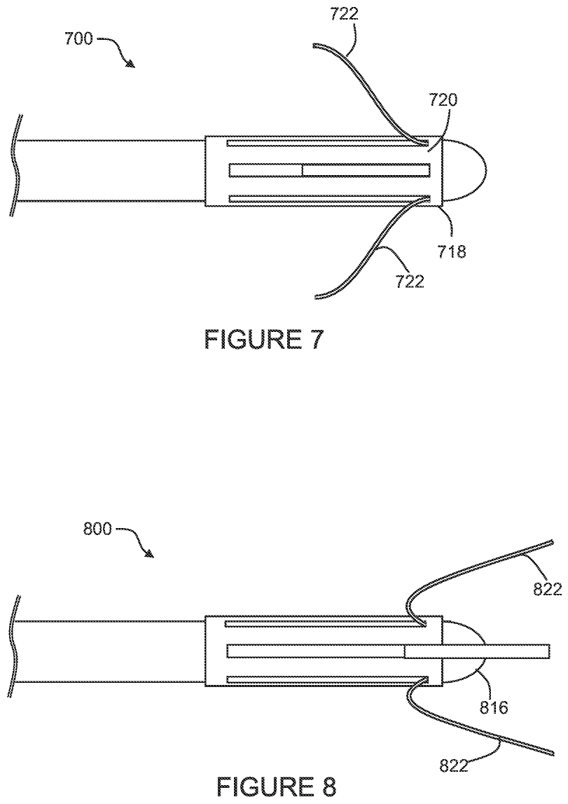

Various configurations of tines are possible. In the example of FIGS. 1 to 6, the ends of the tines 122 remain in contact with the body 120 of the sleeve 118 when the guidewire 100 is in the expanded configuration, so that the tines 122 are generally U-shaped. An alternative example is shown in FIG. 7, in which one end of each tine 722 moves away from the body 720 of the sleeve 718 when the guidewire 700 is in the expanded configuration. A further alternative example is shown in FIG. 8, in which the end of each tine 822 moves distally beyond the electrode 816 when the guidewire 800 is in the expanded configuration. In further alternative examples, when in the expanded configuration, the tines can be straight, wavy, zig-zagged, curved, hooked, or another shape. Furthermore, various numbers of tines can be included, and the tines can be circumferentially spaced apart as shown, and/or longitudinally spaced apart.

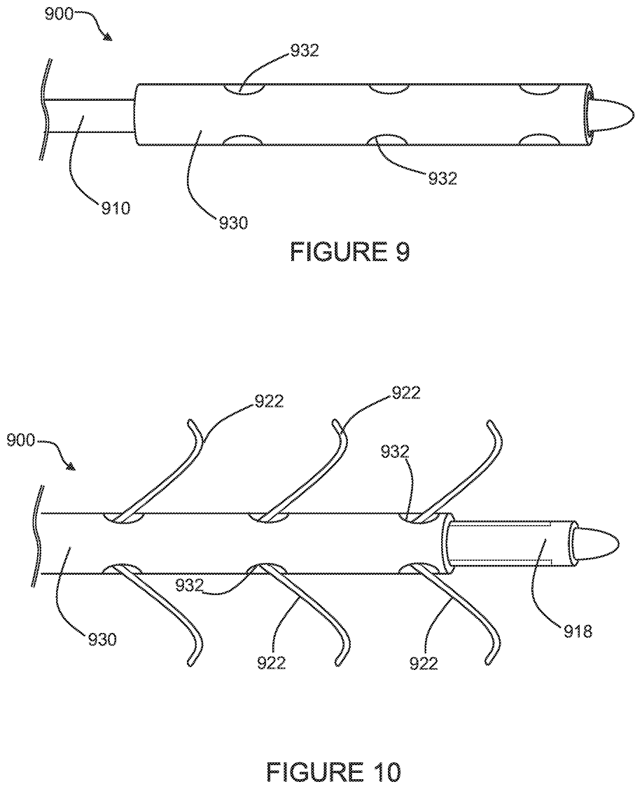

Referring how to FIGS. 9 and 10, an alternative example of a guidewire 900 is shown. In FIG. 9, the guidewire 900 is shown in the retracted configuration, and in FIG. 10, the guidewire 900 is shown in the expanded configuration. In this example, similarly to the examples of FIGS. 1 to 8, the guidewire 900 includes a sleeve 918 (visible in FIG. 10) that is received on a main wire 910 (visible in FIG. 9). The sleeve 918 includes tines 922 (visible in FIG. 10) that flex outwardly when the guidewire 900 is in the expanded configuration, and the tines 922 are biased towards the expanded configuration. However, in this example, the guidewire includes a retainer 930 that is received on the sleeve 918 and is longitudinally slidable in order to hold the tines 922 in the retracted configuration and release the tines to the expanded configuration. More specifically, when the retainer 930 is slid distally, as shown in FIG. 9, the tines 922 are held within the retainer 930. When the retainer 930 slid is slid proximally, as shown in FIG. 10, the tines 922 pass through apertures 932 (only two of which are labelled) in the retainer 930, and flex outwardly away from the sleeve 918. The guidewire 100 can further include an actuating mechanism such as a pull wire (not shown) for sliding the retainer 930.

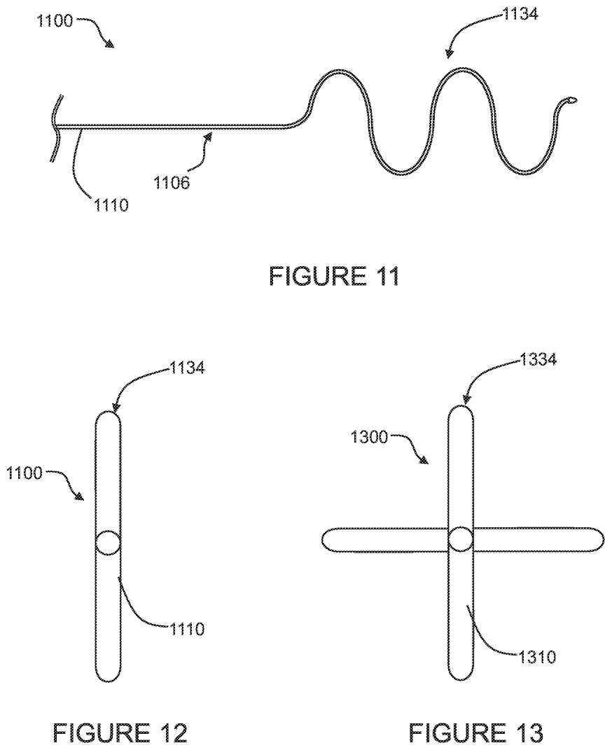

In the above examples, the radially expandable part is in the form of tines. In alternative examples, the main wire itself can be the radially expandable part. For example, in the distal portion, the main wire can include a section that is generally straight when the guidewire is in the retracted configuration, and is serpentine when the guidewire is in the expanded configuration. Moving the wire from the retracted configuration to the expanded configuration can include reshaping the section—i.e. moving the section from a straight shape to a serpentine shape. Similarly to the example of FIGS. 1 to 6, the guidewire can be biased towards the expanded configuration. For example, the electrical conductor of the main wire can be made from a shape memory material such as nitinol. Furthermore, the section can be radiopaque. For example, the electrical conductor can be coated with a radiopaque metal, or the main wire can include a radiopaque band embedded therein, or the electrically insulative material can be filled with a radiopaque filler. In use, the guidewire can be held in the retracted configuration by an introducer. An example of one such a guidewire is shown in FIGS. 11 and 12. In FIGS. 11 and 12, the guidewire 1100 is shown in the expanded configuration. In this example, in the distal portion 1106 of the guidewire 1100, the main wire 1110 includes a section 1134 that is waved when the guidewire 1100 is in the expanded configuration, and is straight when the guidewire 1100 is in the retracted configuration (not shown).

In the example of FIGS. 11 and 12, when the guidewire 1100 is in the expanded configuration, the section 1134 of the main wire 1110 is waved in a single plane—i.e. the waves extend up and down. In alternative examples, when the guidewire is in the expanded configuration, the section of the main wire can be waved in more than one plane. One such example is shown in FIG. 13. In this example, the main wire 1310 of the guidewire 100 includes a section 1134 that is waved in two planes—i.e. the waves extend up and down and side-to-side.

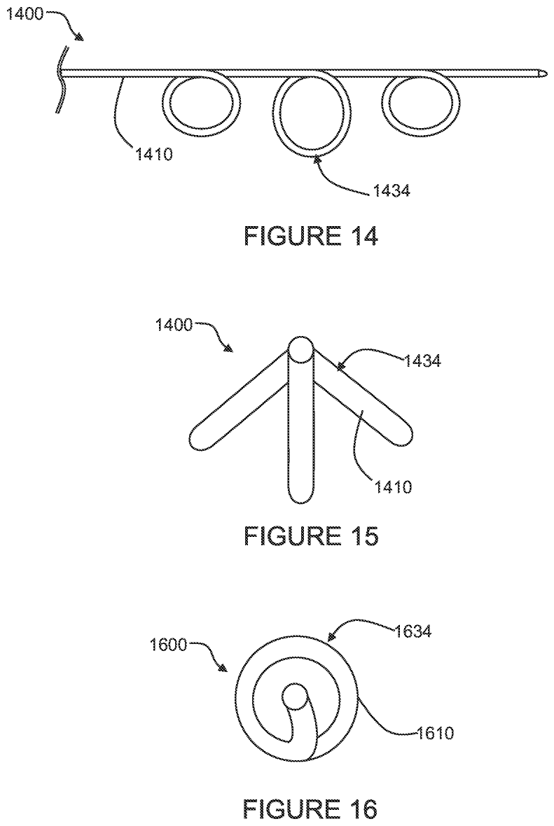

Referring to FIGS. 14 and 15, another example guidewire 1400 is shown. In FIGS. 14 and 15, the guidewire 1400 is shown in the expanded configuration, in which a section 1434 of the main wire 1410 is serpentine. More specifically, when the guidewire 1400 is in the expanded configuration, the section 1434 of the main wire 1410 is coiled. Furthermore, as can be seen in FIG. 15, the coils are oriented in different planes (i.e. can be about 45 degrees apart as in the example shown, or can be about 90 degrees apart) When the guidewire 1400 is in the retracted configuration (not shown), the section 1434 of the main wire 1410 is straight.

Another example guidewire 1600 is shown in FIG. 16, in an expanded configuration. The guidewire 1600 of FIG. 16 is similar to the guidewire 1400 of FIGS. 14 and 15; however, the section 1634 of the main wire 1610 is coiled more tightly, similar to a telephone cord.

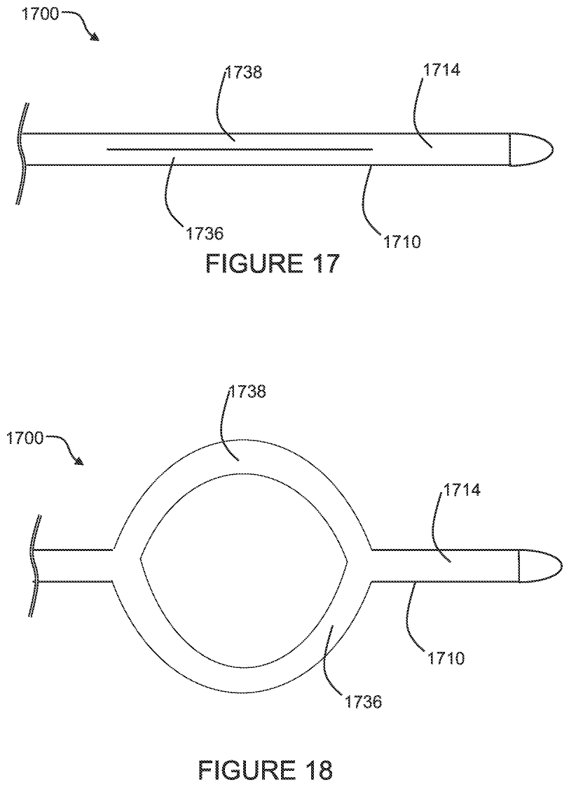

Referring to FIGS. 17 and 18, another example guidewire 1700 is shown, in which the main wire 1710 itself is the radially expandable part. FIG. 17 shows the guidewire 1700 in the retracted configuration, and FIG. 18 shows the guidewire 1700 in the expanded configuration. In this example, the main wire 1710 is split into a first section 1736 and a second section 1738, both of which are radiopaque. When the guidewire 1700 is in the retracted configuration, the first section 1736 and the second section 1738 lie flat against each other. When the guidewire 1700 is in the expanded configuration, the first section 1736 and the second section 1738 are flexed away from each other. Similarly to the above-described examples, the guidewire 1700 can be biased towards the expanded configuration. For example, each section 1736, 1738 of the main wire 1710 can include an electrical conductor (not shown) in the form of a nitinol wire that is biased towards the expanded configuration, and an electrically insulative coating 1714 on the electrical conductor. In order to provide radiopacity, the first 1736 and second 1738 sections can include, for example, a radiopaque metallic coating on the nitinol wire, or a radiopaque filler in the electrically insulative material 1714.

In alternative examples, the guidewire can include more than two sections that are flexed away from each other, or can include additional pairs of sections that are flexed away from each other.

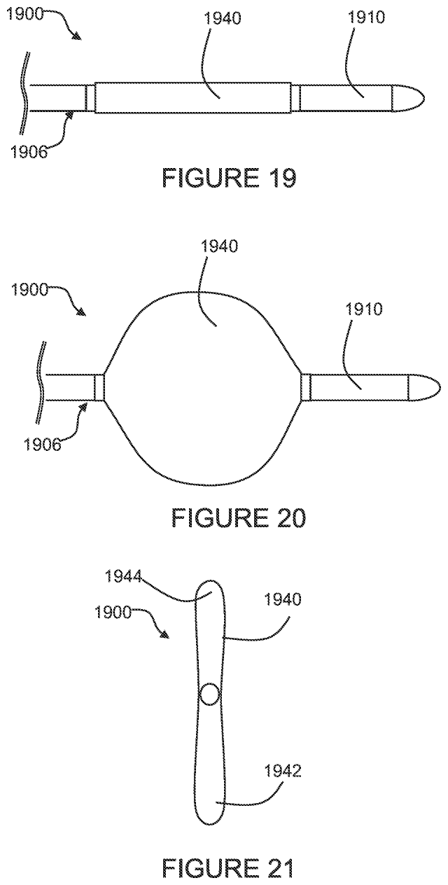

Referring to FIGS. 19 to 21, another example of a guidewire 1900 is shown. In this example, the radially expandable part is in the form of an inflatable balloon 1940 that is mounted to the main wire 1910 in the distal portion 1906. When the guidewire 1900 is in the retracted configuration, as shown in FIG. 19, the balloon 1940 is deflated. When the guidewire 1900 is in the expanded configuration, as shown in FIGS. 20 and 21, the balloon 1940 is inflated. The balloon 1940 can optionally include a radiopaque coating or one or more radiopaque markers. Alternatively, the balloon 1940 can be filled with a contrast medium during inflation. In the example shown, when in the expanded configuration, the balloon 1940 includes a pair of lobes 1942, 1944 (visible in FIG. 21). In alternative examples, when in the expanded configuration, the balloon can include another number of lobes (e.g. three lobes), or can include a single lobe that is centered on the main wire.

In the above examples, the guidewires are configured to deliver RF energy. In alternative examples, guidewires need not be configured to deliver RF energy.

In any of the above examples, the guidewire can include a coiled wire received on the main wire, in order to provide stiffness and/or increase the diameter of the guidewire.

In any of the above examples, the guidewire can include a lubricious coating on the main wire. The lubricious coating can be, for example, polytetrafluoroethylene (PTFE), polyether block amide (PEBA), silicone, nylon, or polyethylene terephthalate (PET).

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A medical device comprising:
a guidewire extending between a proximal portion defining a proximal end and a distal portion defining a distal end;
wherein the distal portion comprises at least one part that is radially expandable, and the guidewire is movable between a retracted configuration in which the part is radially retracted and an expanded configuration in which the part is radially expanded; and
wherein the part is radiopaque.

2. The medical device of claim 1, wherein the part comprises a plurality of tines, wherein when the guidewire is in the retracted configuration, the tines lie generally flat, and when the guidewire is in the expanded configuration, the tines flex radially outwardly.

3. The medical device of claim 2, wherein the tines are circumferentially spaced apart.

4. The medical device of claim 2, wherein:
the guidewire comprises a main wire extending between the proximal portion and the distal portion, and a sleeve on the main wire in the distal portion; and
the sleeve comprises a body and the plurality of tines.

5. The medical device of claim 4, wherein
the guidewire further comprises a retainer on the sleeve, wherein the retainer comprises a plurality of apertures; and
when the guidewire is in the retracted configuration, the tines are held within the retainer, and wherein when the guidewire is in the expanded configuration, the tines extend through the apertures and away from the sleeve.

6. The medical device of claim 4, wherein the main wire comprises an electrical conductor and an electrically insulative material on the electrical conductor, and the guidewire further comprises an electrode at the distal end.

7. The medical device of claim 1, wherein
the guidewire comprises a main wire extending between the proximal portion and the distal portion, and the main wire is the radially expandable part.

8. The medical device of claim 7, wherein when the guidewire is in the retracted configuration, a section of the main wire is generally straight, and when the guidewire is in the expanded configuration, the section is serpentine.

9. The medical device of claim 8, wherein when the guidewire is in the expanded configuration, the section is coiled.

10. The medical device of claim 8, wherein when the guidewire is in the expanded configuration, the section is waved.

11. The medical device of claim 10, wherein when the guidewire is in the expanded configuration, the section is waved in more than one plane.

12. The medical device of claim 7, wherein
in the distal portion, the main wire is split into a first section and a section; and
when the guidewire is in the retracted configuration, the first section and the second section lie flat against each other, and when the guidewire is in the expanded configuration, the first section and the second section are flexed away from each other.

13. The medical device of claim 1, wherein:
the guidewire comprises a main wire extending between the proximal portion and the distal portion, and the part includes an inflatable balloon mounted to the main wire in the distal portion; and
when the guidewire is in the retracted configuration, the balloon is deflated, and when the guidewire is in the expanded configuration, the balloon is inflated.

14. The medical device of claim 1, wherein the part is biased towards the expanded configuration.

15. A method for accessing the pericardial space, comprising:
a. creating a puncture in a pericardium;
b. with a guidewire in a retracted configuration, advancing a distal portion of the guidewire through the puncture into the pericardial space;
c. moving the guidewire to an expanded configuration; and
d. with the guidewire in the expanded configuration and in the pericardial space, viewing the distal portion under fluoroscopy.

16. The method of claim 15, wherein step c. comprises flexing tines of the guidewire radially outwardly.

17. The method of claim 15, wherein step c. comprises reshaping a section of the guidewire.

18. The method of claim 17, wherein step c. comprises moving the section of the guidewire from a straight shape to a serpentine shape.

19. The method of claim 18, wherein step c. comprises moving the section of the guidewire from the straight shape to a coiled shape.

20. The method of claim 18, wherein step c. comprises moving the section of the guidewire from the straight shape to a waved shape.

21. The method of claim 15, wherein step c. comprises flexing a first section of the guidewire away from a second section of the guidewire.

22. The method of claim 15, wherein step c. comprises inflating a balloon of the guidewire.
